(19) United States
(12) Patent Application Publication
(10) Pub. No.: US 2022/0008723 A1
(43) Pub. Date: Jan. 13, 2022

(54) System, Method, and Apparatus for Neurostimulation

(71) Applicant: Unity HA, Effingham, IL (US)

(72) Inventors: William Hsu, Santa Clara, CA (US); Ian Welsford, Concord, CA (US); Mark Van Kerkwyk, Gilroy, CA (US); Vilma Ganesan, Mountain View, CA (US); Bruce Levin, Philadelphia, PA (US)

(21) Appl. No.: 17/327,442

(22) Filed: May 21, 2021

Related U.S. Application Data
(63) Continuation of application No. 16/233,611, filed on Dec. 27, 2018, now abandoned.
(60) Provisional application No. 62/611,254, filed on Dec. 28, 2017.

(51) Int. Cl. A61N 1/36; A61N 1/05; A61N 1/378; A61N 1/372

(52) U.S. Cl. CPC A61N 1/36075; A61N 1/0546; A61N 1/3787; A61N 1/36146; A61N 1/0476; A61N 1/0526; A61N 1/37205; A61N 1/36057; A61N 1/37241

(57) ABSTRACT

An implantable neurostimulator includes a lead comprising a plurality of electrodes at a distal end, and an implant body including electronics for controlling operation of the electrodes. An electrical connector establishes an electrical connection between the electronics and the electrodes. The implant body includes a first portion of the electrical connector, and the proximal end of the lead includes a second portion of the electrical connector. The first and second portions of the electrical connector are connectable to establish the electrical connection between the electronics and the electrodes. The lead is configured for initial implantation in the patient and the implant body is configured for subsequent implantation in the patient. The electrical connector is configured so that the connection of the first and second portions can be performed with the implant body and the lead positioned at a surgical site in the patient.

### TECHNICAL FIELD

The present disclosure relates generally to delivery of energy impulses (and/or energy fields) to bodily tissues for therapeutic purposes and, more particularly, to the use of electrical stimulation of the dorsal nasal nerve structure and other sensory and autonomic nerves for treating disorders in a patient, such as headache or pain.

### BACKGROUND

The sphenopalatine ganglion (SPG) (also known as the pterygopalatine ganglion, ganglion pterygopalatinum, Meckel's ganglion, and nasal ganglion) is a nerve ganglion found in the pterygopalatine (sphenopalatine) fossa, close to the sphenopalatine foramen. The SPG has been a clinical target to treat severe headaches since Sluder first described the application of cocaine or alcohol to the vicinity of the SPG, by swabbing through the nostril to the nasopharyngeal mucosa posterior to the middle turbinate. Unfortunately, the SPG swabbing produces only a brief respite from pain, whether by using a cotton swab as originally described by Sluder, or by means of a topical administration device. In addition, injection into the pterygopalatine fossa (PPF) is difficult to perform reliably due to considerable anatomical variability of the patients, with damage to the maxillary artery that lies next to the SPG being not uncommon. Furthermore, the nasal mucosa may slough during needle insertion. Nevertheless, such pharmacological blockade of the SPG has been claimed to be an effective treatment for headaches, asthma, angina, hiccups, epilepsy, glaucoma, neck pain, vascular spasms, facial neuralgias, blindness, low back pain, sciatica, ear ache, menstrual pain, temporomandibular joint dysfunction, and hyperthyroidism.

More recently, anesthetic has been injected into the PPF using modifications of the Sluder methods and devices. Nevertheless, the internal maxillary artery may be at risk no matter where the PPF is punctured.

In addition to the ganglion blockade using anesthetics as described above, ablation (percutaneous radiofrequency, gamma knife, and surgical ganglionectomy) and electrical nerve stimulation have been used to treat pain (especially cluster headaches) originating in, or emanating from, the SPG. The objective of the ablation is to irreversibly damage the SPG to such an extent that it cannot generate the nerve signals that cause pain. This is not a preferred method because ablation would destroy useful neurophysiological functions of the SPG, notwithstanding the pain that the SPG may cause.

In contrast to ablation, the objective of electrical nerve stimulation is to reversibly damage or otherwise inhibit or block activity the SPG. A significant advantage of electrical stimulation over ablation is that it is a reversible procedure. In that regard, SPG neurostimulation resembles the stimulation of other nerves for the treatment of primary headache disorders.

### SUMMARY

The present disclosure relates generally to delivery of energy impulses (and/or energy fields) to bodily tissues for therapeutic purposes and, more particularly, to the use of electrical stimulation of a dorsal nasal nerve structure, such as a SPG and other sensory and autonomic nerves for treating disorders in a patient, such as headache or pain.

According to one aspect, an implantable neurostimulator includes a lead comprising a plurality of electrodes at a distal end, an implant body comprising electronics for controlling operation of the electrodes and an electrical connector for establishing an electrical connection between the electronics and the electrodes. The implant body comprises a first portion of the electrical connector and the proximal end of the lead comprises a second portion of the electrical connector, the first and second portions of the electrical connector being connectable to establish the electrical connection between the electronics and the electrodes. The lead is configured for initial implantation in the patient and the implant body is configured for subsequent implantation in the patient, and wherein the electrical connector is configured so that the connection of the first and second portions can be performed with the implant body and the lead positioned at a surgical site in the patient.

According to another aspect, alone or in combination with any other aspect, the electrical connector can be a plug-in connector configured so that the electrical connection can be made by pressing together the first and second portions.

According to another aspect, alone or in combination with any other aspect, the first portion of the electrical connector can have a stepped female configuration and the second portion of the electrical connector has a stepped male configuration.

According to another aspect, alone or in combination with any other aspect, the second portion of the connector can be configured for a connection via wires to an external controller. The external controller can be configured to energize the electrodes during implantation in order to obtain feedback for use in positioning the electrodes. The feedback can be feedback indicative of sensing paresthesia induced by the electrodes.

According to another aspect, alone or in combination with any other aspect, the lead can be configured to pass through an 18 gauge surgical needle.

According to another aspect, alone or in combination with any other aspect, the implant body can be configured to pass through a 14 gauge surgical needle.

The implantable neurostimulator can include a remote transducer for providing a wireless signal for powering the stimulator, the remote transducer comprising at least one of a patch, headset, earpiece, extended earpiece, handheld remote controller, headband, and eyeglasses.

According to another aspect, alone or in combination with any other aspect, the implantable neurostimulator can include a remote controller for controlling operation of the remote transducer, the remote controller comprising a foot pedal or a key fob that communicates wirelessly with the remote transducer.

According to another aspect, a method for implanting a two-piece neurostimulator comprising an electrode lead and an implant body connectable with the lead to supply power for energizing the electrodes to apply stimulation therapy includes implanting the lead using a Seldinger technique, implanting the implant body, and connecting the implant body to the lead.

According to another aspect, a method for implanting a two-piece neurostimulator comprising an electrode lead and an implant body connectable with the lead to supply power for energizing the electrodes to apply stimulation therapy to a patient includes attaching a guidewire to the lead, implanting the lead using the guidewire to navigate through the patient's anatomy and position the electrodes at a desired site in the patient, removing the guidewire, leaving the lead implanted, implanting the implant body, and connecting the implant body to the lead.

According to another aspect, a method for implanting a neurostimulator comprising an electrode lead and an implant body for supplying power for energizing the electrodes to apply stimulation therapy to a patient includes attaching a guidewire to the neurostimulator, implanting the stimulator using the guidewire to navigate through the patient's anatomy and position the electrodes at a desired site in the patient, and removing the guidewire, leaving the stimulator.

According to another aspect, a method for implanting a two-piece neurostimulator comprising an electrode lead and an implant body connectable with the lead to supply power for energizing the electrodes to apply stimulation therapy to a patient includes anesthetizing the patient using an anesthesia solution that is sufficient for controlling pain but allows the patient to perceive paresthesia from stimulation, connecting the lead to an external controller that is operable to energize the electrodes to apply stimulation, surgically implanting the lead while applying stimulation via the electrodes, querying the patient for feedback regarding perceived paresthesia during while implanting the lead, using the feedback from the patient to assist in determining a proper position for the lead, securing the lead in the proper position, disconnecting the lead from the external controller, and surgically implanting the implant body and connecting the implant body to the lead.

According to another aspect, alone or in combination with any other aspect, the neurostimulator can be implanted via a gingival-buccal approach, a transoral approach, a trans-nasal a lateral approach through an infratemporal fossa of the patient, or an infrazygomatic approach in which the entry site of the neurostimulator is inferior to the zygoma and anterior to the mandible.

According to another aspect, a method for treating a migraine headache in a patient using an implantable neurostimulator includes programming stimulation parameters into the neurostimulator so that patient does not perceive paresthesia from electrical stimulation of the sphenopalatine ganglion (SPG), implanting the neurostimulator so that a lead of the stimulator having at least one electrode is at a target position proximate to the SPG of the patient, delivering a non-paresthesia stimulation waveform to the at least one electrode based on a therapy parameter set (TPS), the stimulation waveform including a series of pulses configured to excite at least one of A-delta fibers or C-fibers of the SPG of the patient, sensing sensory action potential (SAP) signals of the patient, iterating the steps of delivering the non-paresthesia stimulation waveform and sensing the SAP signals while changing at least one parameter from the TPS, analyzing the SAP signals to obtain SAP activity data associated with the TPS for at least one of an SAP C-fiber component or an SAP A-delta fiber component to obtain a collection of SAP activity data associated with multiple therapy parameter set, selecting one or more parameters for the TPS based on the collection of SAP activity data, programming a pulse generator of the neurostimulator to deliver electrical stimulation to the SPG according to the TPS, activating the neurostimulator so that the pulse generator delivers electrical stimulation to the patient according to the programmed TPS.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a perspective view showing part of the nervous innervation of the anterior craniofacial skeleton;

FIG. 2 is a schematic illustration showing a system for treating a medical condition in a patient constructed in accordance with one aspect of the present disclosure;

FIGS. 3A-3B are schematic illustrations showing a neurostimulator being delivered into a pterygopalatine fossa (PPF) of a patient according to another aspect of the present disclosure; and

FIGS. 4A-4B are schematic illustrations showing the neurostimulator in FIGS. 3A-3B implanted in the PPF and receiving an electrical signal from a remote transducer to treat a medical condition in the subject.

FIGS. 5A-5B are block diagrams illustrating example active/passive variations of the neurostimulator.

FIGS. 6A-6D illustrate an example one-piece configuration of the neurostimulator.

FIGS. 7A-7F illustrate an example two-piece configuration of the neurostimulator.

FIGS. 8A-8E illustrate an example configuration of the neurostimulator including a delivery guidewire.

FIGS. 9A-9L illustrates various different configurations of the remote transducer.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “in communication” can refer to at least a portion of an electrode being adjacent, in the general vicinity, in close proximity, or directly next to and/or directly on (e.g., in physical contact with) a target neural structure, such as a sphenopalatine ganglion (SPG), a sphenopalatine nerve (SPN) (also called the “pterygopalatine nerve”), a vidian nerve (VN) (also called “the nerve of the pterygoid canal”), a greater petrosal nerve (GPN), a lesser petrosal nerve, a deep petrosal nerve (DPN), or a branch thereof (e.g., a nasopalatine nerve, a greater palatine nerve, a lesser palatine nerve, or a superior maxillary nerve). In some instances, the term can mean that at least a portion of an electrode is “in communication” with a target neural structure if application of a therapy signal (e.g., an electrical signal) thereto results in a modulation of neuronal activity to elicit a desired response, such as modulation of a nerve signal (e.g., an action potential or electrical impulse) generated in, or transmitted through, the target neural structure. In such instances, it can be understood that the electrode (or a portion thereof) is in electrical communication with the target neural structure.

A “dorsal nasal nerve structure” includes a SPG, a maxillary nerve, DPN, GPN, VN, nasopalatine nerve, superior posterior lateral nasal branches from the SPG, lesser palatine nerve, greater palatine nerve, and/or an inferior posterior lateral nasal branch from the greater palatine nerve. As used herein with respect to the dorsal nasal nerve structure, the term “electrical communication” refers to the ability of an electric field generated by an electrode to be transferred to the dorsal nasal nerve structure and/or to have a neuromodulatory effect on the dorsal nasal nerve structure. The electrode can be positioned in direct electrical communication with the dorsal nasal nerve structure such that electrode is adjacent to the dorsal nasal nerve structure to directly stimulate the dorsal nasal nerve structure. Such direct electrical stimulation is in contrast to an electrode being placed adjacent to a site distal or proximal to the dorsal nasal nerve structure and thus directly stimulating such distal or proximal sites and indirectly stimulating the dorsal nasal nerve structure. For example, placing an electrode in direct electrical communication with a dorsal nasal nerve structure means that the electrode is not placed adjacent to distal or proximal sites that do or do not innervate the dorsal nasal nerve structure such as, for example, the trigeminal nerve, a branch of the trigeminal nerve, a trigeminal ganglion or the vagus nerve.

As used herein, the term “electrical communication” with respect to a neurostimulator can refer to the ability of an electric field generated by an electrode or electrode array to be transferred, and/or to have a neuromodulatory effect, within and/or on a nerve, neuron, or fiber of a target neural structure.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, etc.

As used herein, the terms “modulate” or “modulating” with reference to activity of a target neural structure can refer to causing a change in neuronal activity, chemistry and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of neuronal activity.

As used herein, the terms “substantially blocked” or “substantially block” when used with reference to activity of a target neural structure can refer to a complete (e.g., 100%) or partial inhibition (e.g., less than 100%, such as about 90%, about 80%, about 70%, about 60%, or less than about 50%) of nerve conduction therethrough. For example, the terms “block”, “blocking”, and “blockade” can refer to the disruption, modulation, and/or inhibition of nerve impulse transmissions through a target neural structure.

As used herein, the term “activity” when used with reference to a target neural structure can, in some instances, refer to the ability of a nerve, neuron, or fiber to conduct, propagate, and/or generate an action potential. In other instances, the term can refer to the frequency at which a nerve or neuron is conducting, propagating, and/or generating one or more action potentials at a given moment in time. In further instances, the term can refer to the frequency at which a nerve or neuron is conducting, propagating, and/or generating one or more action potentials over a given period of time (e.g., seconds, minutes, hours, days, etc.).

As used herein, the terms “prevent” or “preventing” when used with reference to a medical condition (e.g., pain or headache) can refer to stopping a medical condition from occurring, or taking advance measures against the possibility or probability that a medical condition will happen or occur. In some instances, the terms can refer to an action or actions taken to decrease the chance that a subject will contract, develop, or suffer from a medical condition.

As used herein, the terms “suppress” or “suppressing” when used with reference to a medical condition can refer to refer to any quantitatively or qualitatively measurable or observable reduction or attenuation in a medical condition (e.g., a sign or symptom associated with the medical condition).

As used herein, the term “medical condition” can refer to any condition, state, or disease that is characterized, at least in part, by a disruption in sensory signals passing through or associated with the autonomic nervous system (ANS). Non-limiting examples of medical conditions can include pain, autonomic disorders, and neurological disorders. Other examples of medical conditions treatable by the present disclosure can include those disclosed in U.S. Pat. No. 6,526,318 to Ansarinia, U.S. Pat. No. 9,220,524 to Boling et al., U.S. patent application Ser. No. 13/746,038 to Caparso, U.S. patent application Ser. No. 13/917,917 to Goodman et al., U.S. patent application Ser. No. 13/917,953 to Goodman et al., and U.S. patent application Ser. No. 14/093,094 to Pless et al. For example, medical conditions can include headache pain. Headache pain can result from migraine headaches, including migraine headaches with aura, migraine headaches without aura, menstrual migraines, migraine variants, atypical migraines, complicated migraines, hemiplegic migraines, transformed migraines, and chronic daily migraines; episodic tension headaches; chronic tension headaches; analgesic rebound headaches; episodic cluster headaches; chronic cluster headaches; cluster variants; chronic paroxysmal hemicrania; hemicrania continua; post-traumatic headache; post-traumatic neck pain; post-herpetic neuralgia involving the head or face; pain from spine fracture secondary to osteoporosis; arthritis pain in the spine, headache related to cerebrovascular disease and stroke; headache due to vascular disorder; reflex sympathetic dystrophy, cervicalgia (which may be due to various causes, including, but not limited to, muscular, discogenic, or degenerative, including arthritic, posturally related, or metastatic); glossodynia, carotidynia; cricoidynia; otalgia due to middle ear lesion; gastric pain; sciatica; maxillary neuralgia; laryngeal pain, myalgia of neck muscles; trigeminal neuralgia (sometimes also termed tic douloureux); post-lumbar puncture headache; low cerebro-spinal fluid pressure headache; temporomandibular joint disorder; atypical facial pain; ciliary neuralgia; paratrigeminal neuralgia (sometimes also termed Raeder's syndrome); petrosal neuralgia; Eagle's syndrome; idiopathic intracranial hypertension; orofacial pain; myofascial pain syndrome involving the head, neck, and shoulder; chronic migraneous neuralgia, cervical headache; paratrigeminal paralysis; sphenopalatine ganglion neuralgia (sometimes also termed lower-half headache, lower facial neuralgia syndrome, Sluder's neuralgia, and Sluder's syndrome); carotidynia; Vidian neuralgia; and causalgia; or a combination of the above

As used herein, the term “medical condition mediated by autonomic or neurological dysfunction” can refer to any condition, state, or disease that is characterized, at least in part, by a disruption in nerve signals (e.g., action potentials or electrical impulses) passing through or associated with the autonomic nervous system (ANS). Such medical conditions can result from, be caused by (e.g., directly or indirectly), or otherwise be associated with autonomic or neurological dysfunction. Non-limiting examples of medical conditions mediated by autonomic or neurological dysfunction are provided below.

As used herein, the terms “treat” or “treating” can refer to therapeutically regulating, preventing, improving, alleviating the symptoms of, and/or reducing the effects of a medical condition (e.g., mediated by autonomic or neurological dysfunction). As such, treatment also includes situations where a medical condition, or at least symptoms associated therewith, is completely inhibited, e.g., prevented from happening or stopped (e.g., terminated) such that the subject no longer suffers from the medical condition, or at least the symptom(s) that characterize the medical condition.

### Physiological Overview

A brief discussion of the pertinent neurophysiology is provided to assist the reader with understanding certain aspects of the present disclosure.

The autonomic nervous system innervates numerous pathways within the human body and consists of two divisions: the sympathetic and the parasympathetic nervous systems. The sympathetic and parasympathetic nervous systems are antagonistic in their action, balancing the other system's effects within the body. The sympathetic nervous system (SNS) usually initiates activity within the body, preparing the body for action, while the parasympathetic nervous system (PNS) primarily counteracts the effects of the SNS.

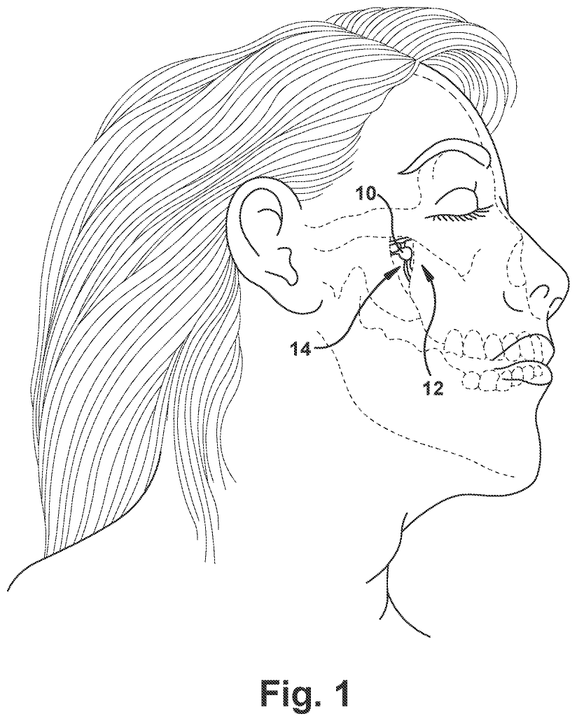

The sphenopalatine ganglia 10 (FIG. 1) are located on both sides of the head. It shall be assumed for the following discussion of the present disclosure that reference is being made to the SPG 10 located on the left side of the head. The SPG 10 is located behind the posterior maxilla 12 in the PPF 14, posterior to the middle nasal turbinate (not shown in detail). The PPF 14 is a small inverted pyramidal space measuring approximately 2 centimeters (cm) high and 1 cm wide and the SPG is approximately 4-5 millimeters (mm) in size. The SPG 10 is part of the parasympathetic division of the autonomic nervous system; however, the SPG has both sympathetic and parasympathetic nerve fibers, as well as sensory and motor nerve fibers either synapsing within the ganglion (e.g., parasympathetic) or fibers that are passing through the ganglion and not synapsing (e.g., sympathetic, sensory, and motor).

The parasympathetic activity of the SPG 10 is mediated through the greater petrosal nerve (not shown), while the sympathetic activity of the SPG is mediated through the deep petrosal nerve (not shown), which is essentially an extension of the cervical sympathetic chain (not shown). Sensory sensations generated by or transmitted through the SPG 10 include, but are not limited to, sensations to the upper teeth, feelings of foreign bodies in the throat, and persistent itching of the ear. The SPG 10 transmits sensory information, including pain, to the trigeminal system via the maxillary division and ophthalmic division (not shown).

The present disclosure relates generally to a system and apparatus for implementing neuromodulatory methods. More particularly, the present disclosure relates generally to a system and apparatus for implementing neuromodulatory methods for treating medical conditions by stimulation of a target neural structure. As discussed in more detail below, the system and apparatus can be used to implement methods for suppressing or preventing medical conditions by disrupting sensory signals passing through the ANS, such as pain signals. The abnormal regulation of pain or autonomic pathways, which may be a feature of the medical conditions disclosed herein, can cause excitation, loss of inhibition, suppression, or loss of excitation of these pathways. Thus, in some instances, the system and apparatus can be used to implement methods for applying one or more electrical signals to a target neural structure in order to modulate the transmission of sensory signals and stimulate or block the autonomic pathways passing through the target neural structure to reduce or eliminate one or more symptoms or signs associated with a medical condition. In other instances, the application of one or more electrical signals to a target neural structure can modulate the transmission of sensory signals other than pain responsible for provoking or aggravating other undesirable sensations or conditions, such as nausea, bladder disorders, sleep disorders or abnormal metabolic states.

### System Overview

According to one aspect, the present disclosure relates to a system 16 (FIG. 2) for preventing, suppressing, or treating a medical condition in a patient. The components of the system 16 can generally include a neurostimulator 18 and a remote transducer 20. The system 16 can also include a personal electronic device 22 and/or programming device 24. As discussed below, each component of the system 16 can be in communication (e.g., electrical communication) with one another. In some instances, two or more components of the system 16 can be in wireless communication with one another. In other instances, two or more components of the system 16 can be in wired communication with one another. It will be appreciated that some components of the system 16 can be in wireless communication with one another while other components are in wired communication with one another.

The neurostimulator 18 can be sized and dimensioned for injection or insertion into a PPF 14 of a patient. The neurostimulator 18 can comprise electronic circuitry (not shown), one or more electrodes (not shown) that is/are driven by the circuitry, and one or more transmit coils, radiators, or PCB antennas (not shown). The electronic circuitry of the neurostimulator is programmed to receive and transmit data (e.g., stimulation parameters) and/or power from outside the body. In some instances, the electronic circuitry can be encapsulated by an insulative, biocompatible resin. The neurostimulator 18 can be entirely or partly formed from a flexible, biocompatible polymer. In some instances, the electronic circuitry can include a programmable memory for storing at least one set of stimulation and control parameters. In other instances, the neurostimulator 18 can include a power source (not shown) and/or power storage device (not shown). Possible power options can include, but are not limited to, various wireless charging mechanisms, such as an external power source coupled to the neurostimulator via an RF link using coils or radiators, a self-contained power source utilizing any means of generation or storage of energy (e.g., a primary battery, a rechargeable battery, such as a lithium ion battery, a button or coin cell battery, an electrolytic capacitor, or a super- or ultra-capacitor), and, if the self-contained power source is rechargeable, a mechanism for recharging the power source (e.g., an RF link). In some instances, the system 16 can include a retractable power cable (not shown) that can be selectively connected to the power source and/or power storage device.

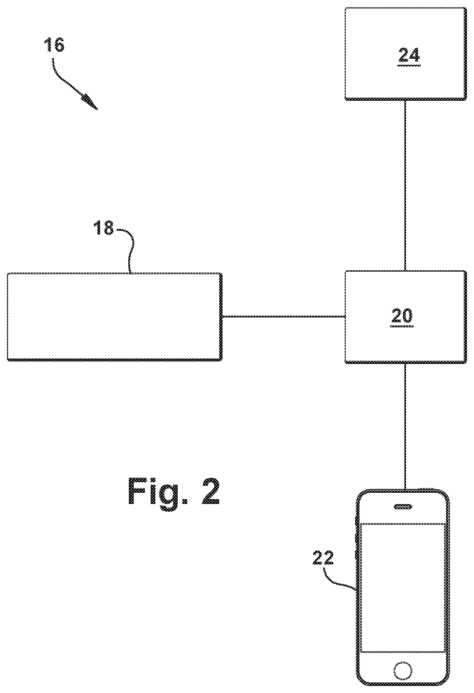

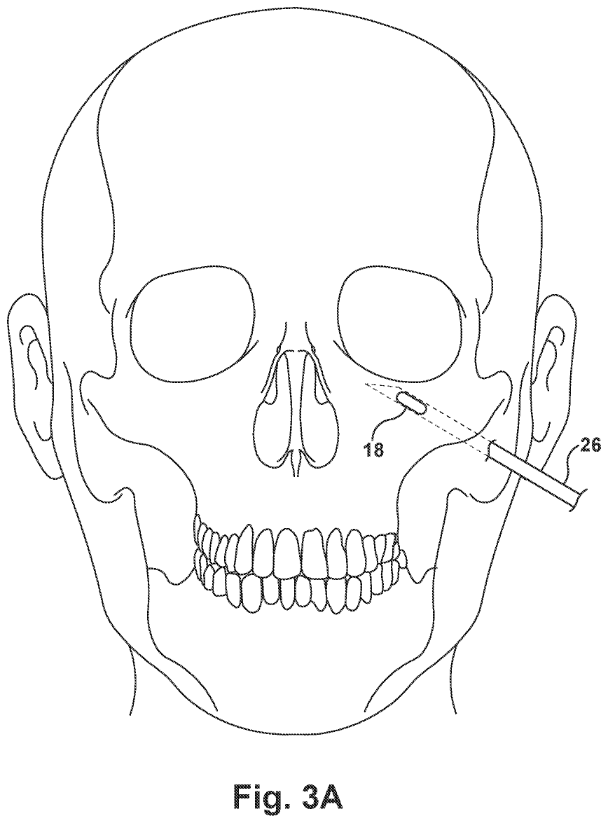

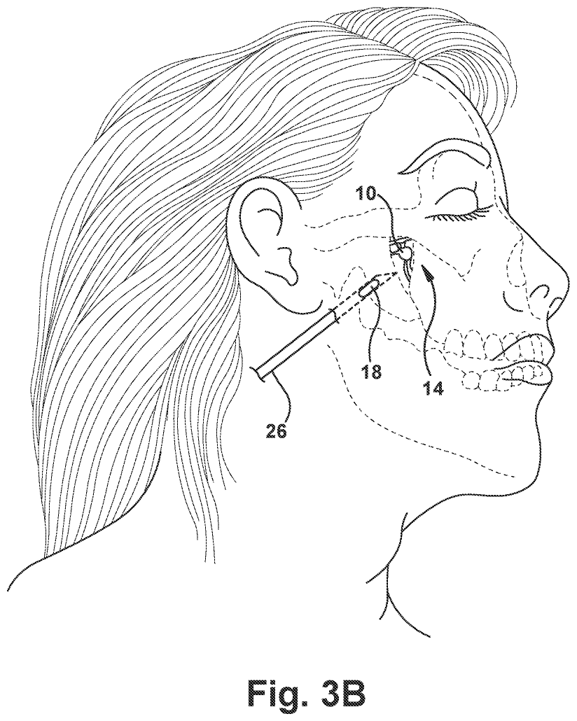

The neurostimulator 18 can be sized and dimensioned for injection or insertion into the PPF 14 via an elongated, hollow, tubular delivery device 26 (FIGS. 3A-3B). The delivery device 26 can, for example, be configured to possess sufficient lubricity to promote passage of the neurostimulator 18 through its inner lumen and to prevent damage during delivery. The delivery device 26 can comprise, for example, a needle, a catheter, or a catheter-like device. One particular example of such a delivery device 26 is a 12-20 gauge needle. A 14-gauge needle can be useful because it has an outside diameter small enough to permit navigation in a minimally invasive manner, and an inner diameter sufficiently large enough to facilitate delivery of the neurostimulator or portions thereof. In one example, the delivery device 26 can be configured to include a connection (not shown) within the delivery device for establishing an electrical connection with the neurostimulator 18 to allow stimulation and response profiling during implantation. Additionally, the delivery device 26 can be configured to include navigation features for facilitating placement of the neurostimulator, such as a steerable tip (not shown).

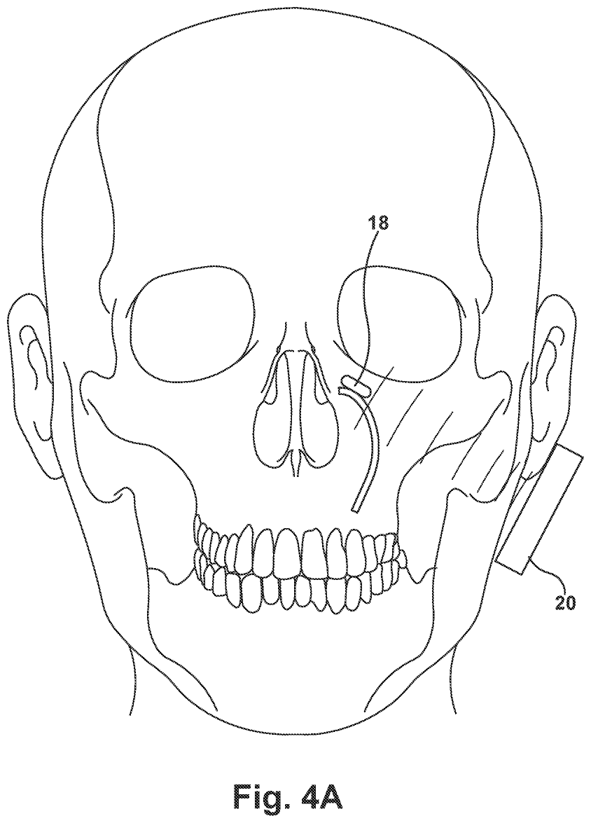

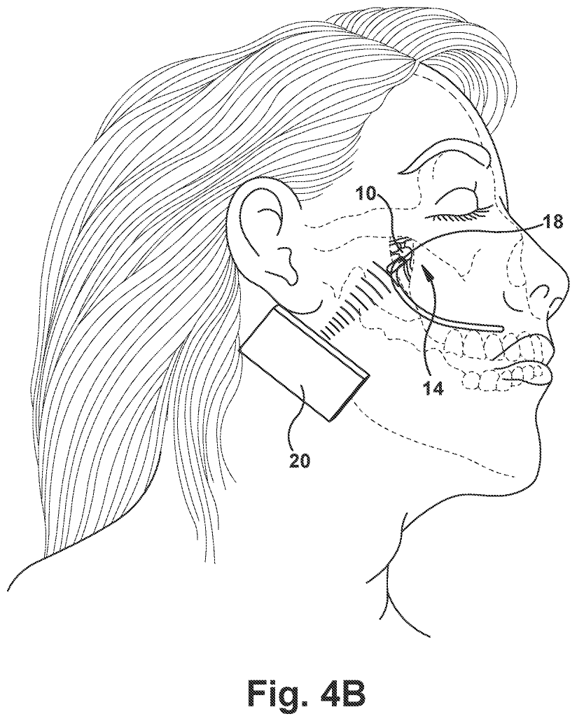

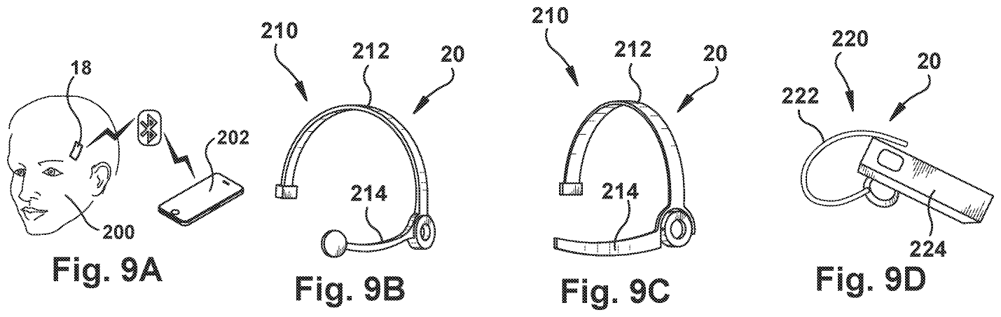

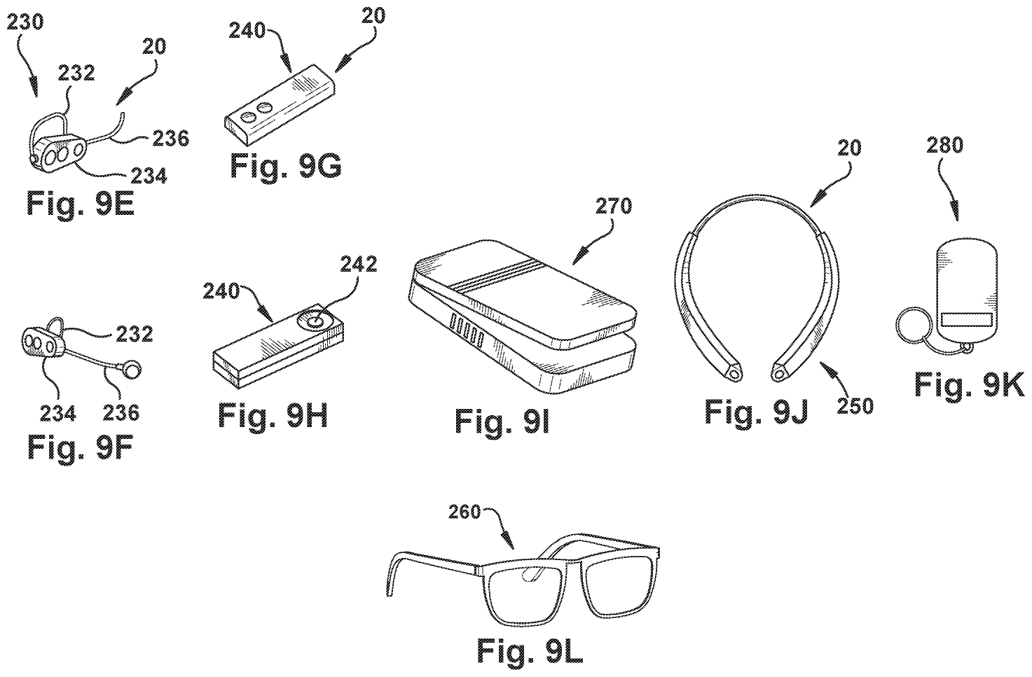

According to another aspect, referring to FIGS. 2, 4A, and 4B, the system 16 can include a remote transducer 20 in electrical communication (e.g., wireless communication) with the neurostimulator 18. The remote transducer 20 can be programmed and configured for delivery of an electrical signal to the neurostimulator 18. In some instances, the remote transducer 20 can comprise a replaceable or rechargeable power source (not shown) and a transmit coil (not shown), each of which is partly or entirely located within a housing (not shown). The remote transducer 20 is adapted for placement on or about a patient's head, e.g., adjacent an implanted neurostimulator 18 of the present disclosure. FIGS. 4A-4B illustrate a remote transducer 20 in the form of a patch. The remote transducer can, however, have alternative configurations, such as a wand, glasses, or any of the other example configurations illustrated in FIGS. 9A-9L, which is discussed below. In such instances, the remote transducer 20 can be programmed to provide user feedback to assist the subject in optimizing placement of the transducer about the subject's body. Where the remote transducer 20 is configured as a patch, at least one skin-contacting surface of the patch can include an adhesive coating or other material (e.g., a hydrogel) to permit attachment of the patch to a skin surface (e.g., the cheek) of a patient. Additionally, where the remote transducer 20 is configured as a patch, the patch can be adapted to fit on replacement skin patches. The patch can also include such components as a rechargeable battery, Bluetooth capability, and closed-loop control circuits (described more below).

In another aspect, the system 16 can include a personal electronic device 22 that is in electrical communication (e.g., wireless communication) with the remote transducer 20. Examples of personal electronic devices 22 can include smart phones and tablets, although it will be appreciated that personal computers (PCs) may also be included. In some instances, the personal electronic device 22 can include software programmed to control one or more stimulation and/or control parameters associated with the neurostimulator. Additionally, or optionally, the software comprising the personal electronic device 22 can be programmed to store patient therapy data, such as diary questions and patient incentive information, and/or promote patient-to-patient interaction. For instance, the personal electric device 22 can be programmed to include an electronic leader board where patients are ranked against other patients based on certain usage goals. The personal electronic device can also be programmed to interact with an incentive program for patients to earn “points” for compliance (e.g. activating the device once every day for 20 minutes) so that a manufacturer could study new therapies or gather product data. The personal electronic device 22 can also include software programmed to access remote data sources (e.g., Internet websites), query certain data, and then provide stimulation instructions to the system 16 based on the queried data. For example, the personal electronic device 22 can access a website that provides weather-related information (e.g., Accuweather) and then, based on information obtained from the website, provide predictive information and/or stimulation instructions for a particular medical condition (e.g., migraine). In another example, the personal electronic device 22 can also include software programmed to provide the neurostimulator 18 with customizable or patient-triggered alerts, e.g., indicating stimulation periods and the duration of each period, after a desired period of time (e.g., 1.5 hours) after sleep onset, or after consumption of food or water. In some instances, the personal electronic device 22 can be operated manually by the patient or a caregiver.

In another aspect, the system 16 can additionally or optionally comprise a programming device 24 that is in electrical communication (e.g., wireless communication) with the remote transducer 20. The programming device 24 can be configured and programmed to deliver stimulation and/or control instructions to the remote transducer 20. In one example, the programming device 24 can be configured as a dedicated, smart phone-sized unit. In another example, the programming device 24 can be configured as a smart phone accessory dongle. In some instances, the programming device 24 can be operated manually by the patient or a caregiver. In other instances, the programming device 24 can be battery powered and/or directly powered, e.g., by an AC source. If powered by rechargeable batteries, a battery charger may be an accessory to the programming device 24.

In another aspect, the system 16 can include one or more sensors (not shown) to permit open-loop or closed-loop control. In an open-loop system, for example, the system 16 can include one or more sensors such that a patient can manage (e.g., prophylactically) treatment of a medical condition based on feedback (e.g., detected signals) from the sensor(s). Such detected signals can be indicative of the onset of a medical condition, such as an increase in blood flow, skin resistance, temperature, etc. Upon noticing the signal(s), the patient can then trigger or activate the neurostimulator 18 to prevent or mitigate onset of the medical condition.

In another aspect, the system 16 can include one or more sensors to permit closed-loop control by, for example, automatically responding (e.g., by activation of the neurostimulator 18) in response to a sensed environmental parameter and/or a sensed physiological parameter, or a related symptom or sign, indicative of the extent and/or presence of a medical condition. In one example, the sensor(s) can detect an environmental parameter, such as barometric pressure, ambient temperature, humidity, etc. In another example, the sensor(s) can detect a physiological parameter, or a related symptom or sign, indicative of the extent and/or presence of a medical condition, non-limiting examples of which include skin resistance, blood flow, blood pressure, a chemical moiety, nerve activity (e.g., electrical activity), protein concentrations, electrochemical gradients, hormones (e.g., cortisol), electrolytes, markers of locomotor activity, and cardiac markers (e.g., EKG RR intervals). Sensors used as part of a closed-loop or open-loop system can be placed at any appropriate anatomical location on a subject, including a skin surface, an intra-oral cavity, a mucosal surface, or at a subcutaneous location. Examples of sensors and feedback control techniques that may be employed as part of the present disclosure are disclosed in U.S. Pat. No. 5,716,377.

### Neurostimulator/Remote Transducer Configurations

From the above, it will be appreciated that the system 16 includes an implantable portion or part comprising the neurostimulator 18 and an external portion or part comprising the remote transducer 20 for powering and/or communicating with the neurostimulator. The system 16 can be configured for active or passive stimulation. In an active stimulation configuration, the neurostimulator includes hardware configured to store at least some parameters/settings, and to control activation of the electrodes in order to apply stimulation therapy according to the stored parameters/settings. In a passive stimulation configuration, the neurostimulator is configured via hardware to activate the electrodes in a predetermined manner in response to the excitation signal received form the remote transducer.

Active Neurostimulator Configuration

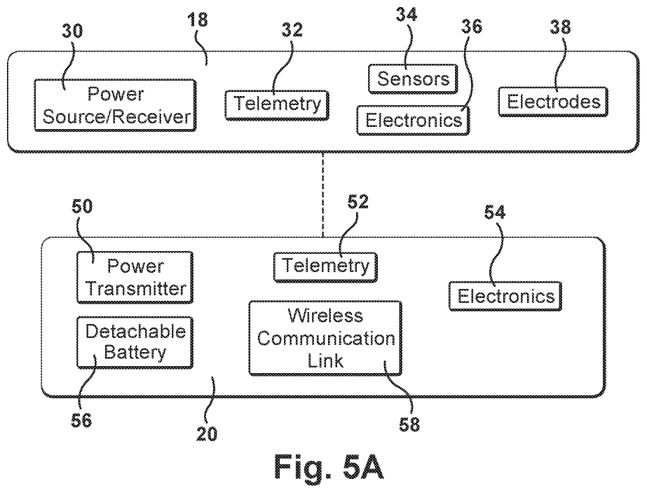

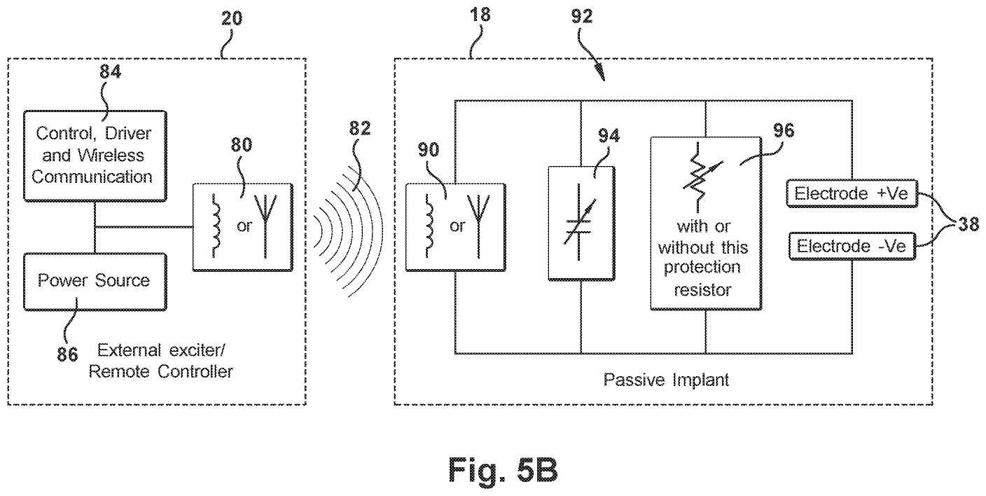

Referring to the block diagram of FIG. 5A, the remote transducer 20 includes a power transmitter portion 50, a telemetry portion 52, an electronics portion 54, a battery portion 56, and a wireless communication link portion 58.

The power transmitter portion 50 transmits power using wireless power transfer technologies mated to the power portion 30 of the neurostimulator 18. The power transmitter portion 50 includes a transmission element, such as a coil or antenna, for generating a wireless power transfer signal, such as an RF power transfer signal. The power transmitter portion 50 can therefore be configured to transmit power to the neurostimulator 18 via wireless power transfer technologies, such as inductive coupling, inductive resonate magnetic coupling, capacitive coupling, near-field coupling, mid-field coupling, far-field coupling, microwave power, ultrasonic/acoustic power, and light power.

One particular wireless power transfer technology that can be implemented in the remote transducer 20 is microwave RF power transfer technology. Microwave RF power transfer operates at 2-10 GHz and is highly efficient and can be implemented using a comparatively small form factor antenna. Additionally, microwave power transfer does not pose any directivity issues, so the orientation and position of the remote transducer relative to the neurostimulator 18 can be more generalized.

The telemetry portion 52 communicates via an RF communication protocol mated to the telemetry portion 32 of the neurostimulator 18. The telemetry portion 52 can therefore be configured to communicate via can communicate via any appropriate radio frequency communication protocol.

The electronics portion 54 can be configured to perform control functions, processing functions, power management functions, and telemetry functions to control communications with the neurostimulator 18 and external devices, such as a personal electronic device 22 via Bluetooth, Wi-Fi, etc.

The battery portion 56 can be detachable for swapping and prolonged usage. A swappable battery portion 56 can be disposable or rechargeable. An example battery portion is a rechargeable lithium-ion battery.

The wireless communication link portion 58 performs the communication with the neurostimulator 18 under the direction of the telemetry control performed by the electronics portion 54. The wireless communication link portion 58 can, for example, include a Bluetooth radio and/or a Wi-Fi radio.

Also, referring to the block diagram of FIG. 5A, the neurostimulator 18 can include a variety of components, including a power portion 30, a telemetry portion 32, a sensor portion 34, an electronics portion 36, and an electrodes portion 38. These portions are illustrated individually in FIG. 5A only to show the different functions that take place on the neurostimulator 18. These portions are not necessarily discrete portions or components of the neurostimulator device itself, as several of the functions illustrated by these different portions can be implemented on the same hardware device or component.

The power portion 30 provides electrical power to the neurostimulator components that require it. The power portion 30 can be any of the following, individually or in combination: wireless power, battery power, and charge banks. When the power portion 30 includes wireless power, it is configured to receive power from the remote transducer via wireless power transfer technologies, such as inductive coupling, resonate inductive coupling, capacitive coupling, near-field coupling, mid-field coupling, far-field coupling, microwave power, ultrasonic/acoustic power, and light power. When the power portion 30 includes battery power, the batteries can be disposable batteries, such as nickel-cadmium batteries or rechargeable batteries, such as lithium-ion batteries. When the power portion 30 includes charge banks, the charge banks can include capacitors, inductors, and super-capacitors, which can be used on a standalone basis or in combination with the battery and/or wireless power.

The telemetry portion 32 can communicate via any radiofrequency (RF) technology for communicating with the remote transducer 20.

The sensor portion 34 can be any sensor used to sense the sphenopalatine ganglion (SPG) nerve bundle for closed loop feedback control.

The electronics portion 36 can be configured to perform control functions, processing functions, power management, telemetry control, and stimulation control.

The electrodes portion is the portion that establishes electrical contact with the sphenopalatine ganglion (SPG) nerve bundle to deliver the stimulation current to the SPG nerve bundle.

Passive Neurostimulator Configuration

Referring to the diagram of FIG. 5B, the remote transducer 20 includes a coil or antenna 80 for transmitting a wireless control/power signal 82 (e.g., electromagnetic, RF, microwave, etc.) to the remote transducer 18. The neurostimulator 18 includes a coil or antenna 90 that receives and is excited by the wireless control/power signal 68 transmitted by the remote transducer 20. The coil/antenna 90 forms part of a stimulator circuit 92. The stimulator circuit 92 also includes a charge circuit 94 that includes one or more charge storage capacitors, and an optional protection circuit 96 that includes one or more protection resistors. The stimulator circuit 92 also includes, of course, the electrodes 38.

The remote transducer 20 can have the same general configuration as those described above with reference to FIGS. 4A, 4B, and 5A. for purposes of illustrating the passive neurostimulator configuration of FIG. 5B, the configuration of the remote transducer 20 is generalized so as to include a control portion 84 including control, driver, and wireless communication electronics, a power source 86, and the antenna 80. The control portion 84 excites the coil/antenna 80, using power from the power source 86, to regulate/modulate the transmitted signal 82 according to parameters entered or otherwise programmed into the remote transducer 20. Upon activation, the remote transducer 20 generates the wireless control/power signal 82 to cause operation of the implanted neurostimulator 18. The coil/antenna 90 of the neurostimulator 18 receives and is excited by the signal 82, and this excitation generates an induced current that is supplied to the stimulator circuit 92. This current charges the storage circuit 94, which provides the power for applying stimulation via the electrodes 38.

According to the passive neurostimulator configuration of FIG. 5B, the capacitance of the charge circuit 94 is configured to tune the stimulator circuit 92 to energize the electrodes 38 with a sinusoidal waveform that is predetermined to cause the passive stimulator 18 to apply a desired stimulation therapy regimen. According to one example, the capacitance of the charge circuit 94 can be configured to tune the stimulation circuit 92 to apply stimulation therapy with a sinusoidal waveform at a frequency in the range of 1 Hz to 150 kHz. The passive stimulator antenna 90 is excited by the control/power signal 82, generating a current that drives the stimulator circuit 92. The capacitor(s) of the charge circuit 94 accumulate a charge, which is discharged at the rate or frequency dictated by the circuit capacitance. The stimulator circuit 92 energizes the electrodes 38 and applies stimulation therapy in accordance with this waveform. Since the capacitance of the charge circuit 94 is tuned to correspond to a desired therapy waveform, it can be seen that the stimulator 18 can apply stimulation therapy passively, according to a desired therapy regimen.

### Neurostimulator Designs

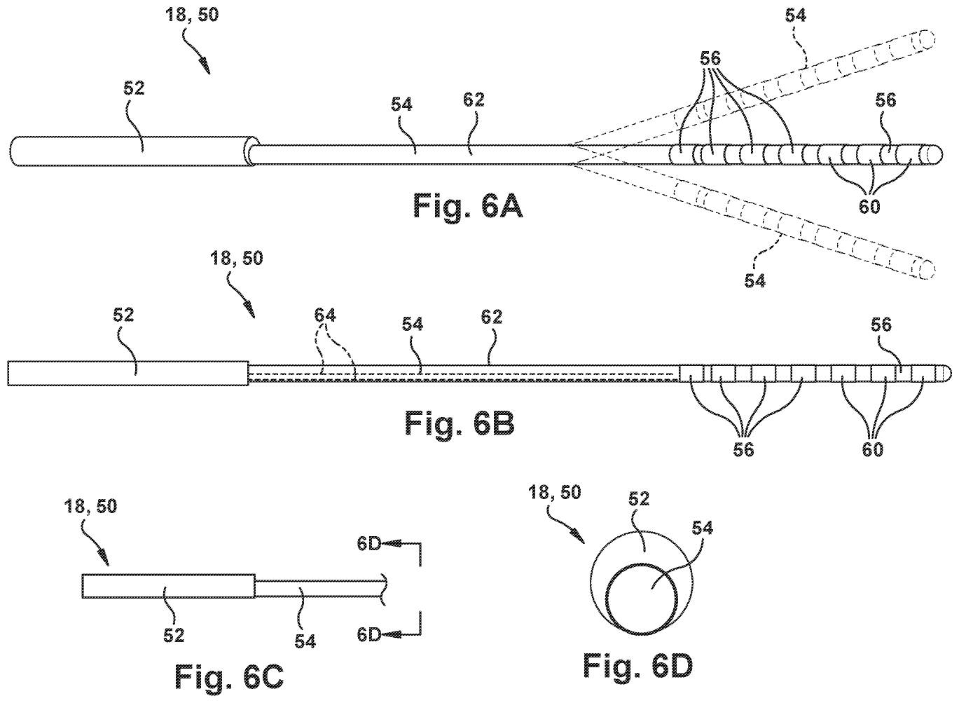

The design/configuration of the neurostimulator 18 can vary. Example configurations are shown in FIGS. 6A-6D, FIGS. 7A-7E, and FIGS. 8A-8E.

One-Piece Neurostimulator Design

According to one aspect, the neurostimulator 18 can have a single component configuration. Referring to FIGS. 6A-6D, according to one example single component configuration, the neurostimulator 18 can be a one-piece neurostimulator 50 configured to fit into a surgical needle for delivery via injection or insertion. In one particular example configuration, the entire neurostimulator 18 can fit within or pass through a 14-gauge needle. As shown in FIGS. 6A-6D, the neurostimulator 50 includes an implant body 52 and a lead 54 connected to the implant body. The neurostimulator 50 also includes a plurality of electrodes 60 disposed at the distal end 56 of the lead 54.

The implant body 52 includes the electronic components necessary to perform the various functions for applying stimulation therapy via the electrodes 60. These components can include, for example, application specific integrated circuits (ASICs), custom field programmable gate array (FPGA) chips, a system on a chip (SoC), an integrated circuit (IC) with additional components assembled in a ceramic package, or a combination thereof. In one particular configuration, the implant body 52 can include an application specific integrated circuit (ASIC) with discrete components, such as antennas/coils, capacitors, resistors, etc., for power transmission, distribution, and control. The lead 54 includes a lead body 62 and lead wires 64 that extend through the lead body and electrically connect the electronics of the implant body 52 to the electrodes 60. The lead wires 64 can extend through the lead body 62, for example, by passing through an inner lumen of the lead (i.e., the lead body 62 can have a tubular construction) or by being embedded within the lead body material (e.g., the lead body can have a solid construction).

The lead body 62 and lead wires 64 have a configuration and material construction selected such that the lead 54 can be both flexible and deformable. This bending is shown by way of example in dashed lines in the figures. As a result, the lead 54 can be bent or otherwise physically manipulated to a shape that is maintained once released. For example, the deformable characteristics of the lead 54 can be created through the metal material used to form the lead wires 64. The material used to construct the lead body 62 can be a flexible, conforming material, such as a soft plastic or polymer, that adopts the shape to which the metal lead wires 64 are bent or otherwise deformed. The metal used to form the lead wires 64, can be selected to have a ductility such that the lead wires can maintain the shape into which they are bent or otherwise deformed. The lead wires 64 can, for example, be constructed of solid copper wire (as opposed to stranded wire).

The lead 54 can be bent to follow the anatomy of the patient, allowing the electrodes 60 to be positioned at a desired position and orientation relative to the SPG. This also allows the implant body 52 to be positioned in a location that is least intrusive to the patient in terms of discomfort and/or visibility (e.g., externally visible lumps), if applicable. The lead 54, following or conforming to the patient anatomy, can help maintain the entire stimulator 50 in the desired implanted position/orientation.

Two-Piece Neurostimulator Design

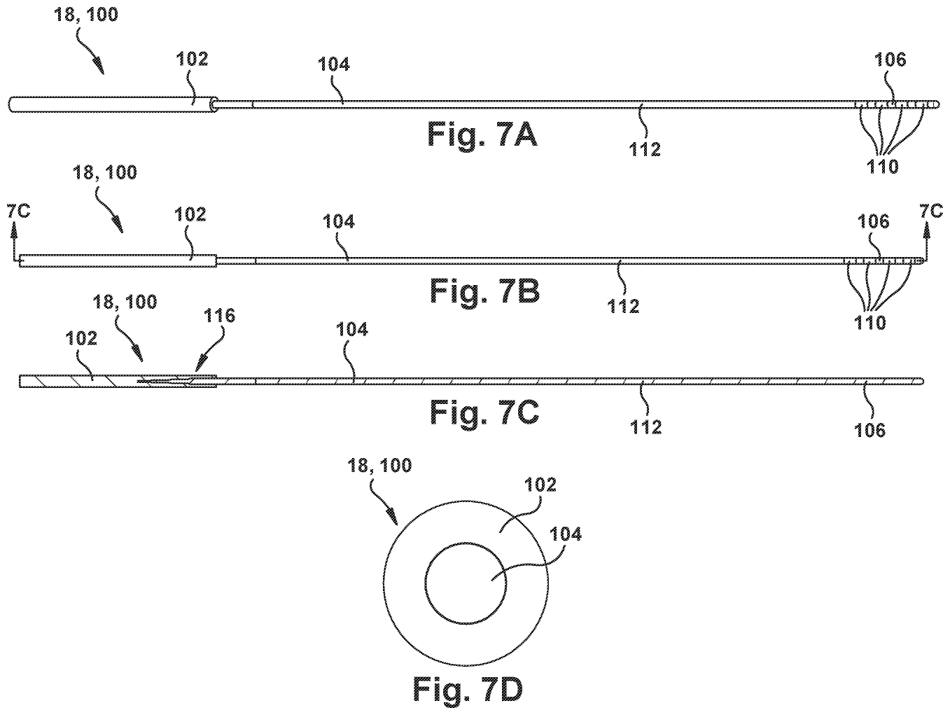

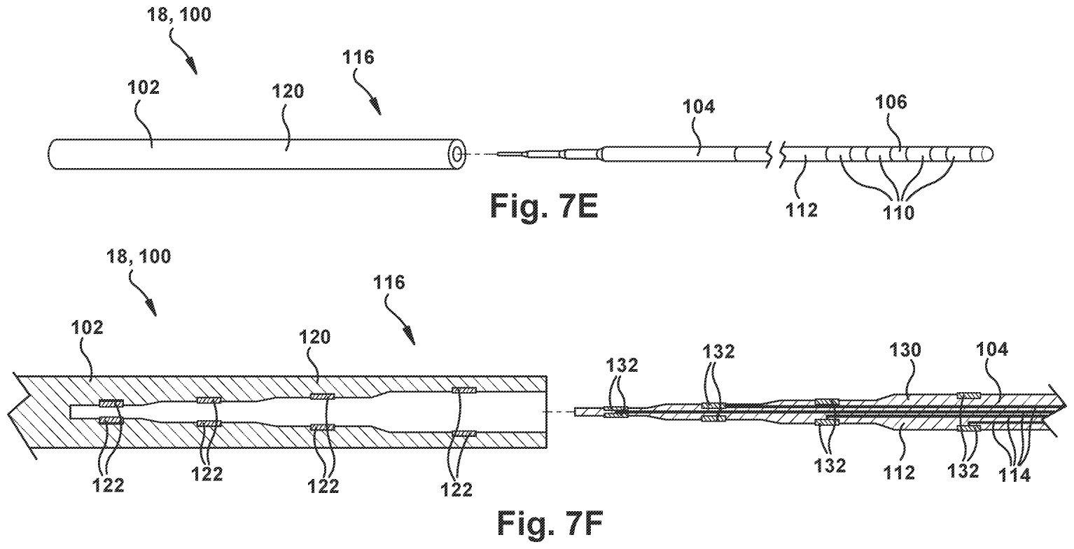

According to another aspect, the neurostimulator 18 can have a multiple component configuration. Referring to FIGS. 7A-7E, according to one example multiple component configuration, the neurostimulator 18 can be a two-piece neurostimulator 100 configured to fit into a surgical needle for delivery via injection or insertion. As shown in FIGS. 7A-7E, the neurostimulator 100 includes two portions—an implant body 102 and a lead 104, that are detachably connected to each other. The neurostimulator 100 also includes a plurality of electrodes 110 disposed at the distal end 106 of the lead 104.

The implant body 102 includes the electronic components necessary to perform the various functions for applying stimulation therapy via the electrodes 110. The implant body 102 can include an application specific integrated circuit (ASIC) with discrete components, such as antennas/coils, capacitors, resistors, etc., for power transmission, distribution, and control. The lead 104 includes a lead body 112 and lead wires 114 that extend through the lead body and electrically connect the electronics of the implant body 102 to the electrodes 110. The lead wires 114 can extend through the lead body 112, for example, by passing through an inner lumen of the lead (i.e., the lead body 112 can have a tubular construction) or by being embedded within the lead body material (e.g., the lead body can have a tubular or solid construction).

The neurostimulator 100 includes a connector 116 for facilitating the detachable connection between the implant body 102 and the lead 104. Referring to FIGS. 7A-7E, the connector 116 includes a first portion 120 on the implant body 102 and a second portion 130 on the lead 104. In the example configuration illustrated in FIGS. 7A-7E, the first portion 120 is a female connector configured to receive, mate with, and retain the second portion 130, which is a male connector. The male second portion 130 has a stepped outside diameter configuration that mates with a stepped inside diameter configuration of the female first portion 120. This stepped configuration helps ensure proper alignment and engagement between electrical contacts of the first and second portions 120, 130.

The first portion 120 includes electrical contacts 122 that engage corresponding electrical contacts 132 on the second portion 130 when the first and second portions of the connector 116 are interconnected with each other. The electrical contacts 122 of the first portion 120 are electrically connected to the electronics of the implant body 102. The electrical contacts 132 of the second portion 130 are electrically connected to the electrodes 110 via the lead wires 114. The number of electrical contacts 122, 132 provided on the first and second portions 120, 130 can correspond to the number of electrodes on the lead 104. For instance, in the example configuration of the stimulator 100 illustrated in FIGS. 7A-7E, the stimulator 100 includes four electrodes 110, so the first portion 120 of the connector 116 would include four contacts 122, and the second portion 130 of the connector would include four contacts 132. From this, it can be seen that electrical continuity can be established between the electronics of the implant body 102 and the electrodes 110 via the connector 116 and the lead wires 114.

The lead body 112 and lead wires 114 can have a configuration and material construction selected such that the lead 104 can be both flexible and deformable. As a result, the lead 104 can be bent or otherwise physically manipulated to a shape that is maintained once released. For example, the deformable characteristics of the lead 104 can be created through the metal material used to form the lead wires 114. The material used to construct the lead body 112 can be a flexible, conforming material, such as a soft plastic or polymer, that adopts the shape to which the metal lead wires 114 are bent or otherwise deformed. The metal used to form the lead wires 114, can be selected to have a ductility such that the lead wires maintain the shape into which they are bent or otherwise deformed. The lead wires 114 can, for example, be constructed of solid copper wire (as opposed to stranded wire).

The lead 104 can be bent to follow the anatomy of the patient, allowing the electrodes 110 to be positioned at a desired position and orientation relative to the SPG. This also allows the implant body 102 to be positioned in a location that is least intrusive to the patient in terms of discomfort and/or visibility (e.g., externally visible lumps), if applicable. The lead 104, following or conforming to the patient anatomy, can help maintain the entire neurostimulator 100 in the desired implanted position/orientation.

The two-piece configuration of the neurostimulator 100 can allow for implanting the lead 104 separately from the implant body 102. Since the lead 104 can be configured to have a diameter that is smaller than the implant body 102, implanting the lead separately can allow for it to be delivered using a smaller diameter device, such as a surgical needle. The implant body 102, having a larger diameter, can be delivered with a larger diameter device/needle. For instance, in one example configuration, the implant body 102 can be configured for delivery via a 14 gauge needle and the lead 104 can be configured for delivery via an 18 gauge needle. Delivering the lead 104 with a smaller needle can offer greater dexterity and can reduce the invasiveness of the procedure, and the discomfort and pain felt by the patient.

Additionally, the connector 116 can facilitate connecting the lead 104 to an external device, which enables actuation of the electrodes 110 without requiring the use of the remote transducer 20. Because of this, an external device, such as a controller, can be wired directly to the lead 104 and used to apply stimulation via the electrodes 110 in order to assist in finding the proper placement of the lead 104.

Implantation Device-Free Neurostimulator Design

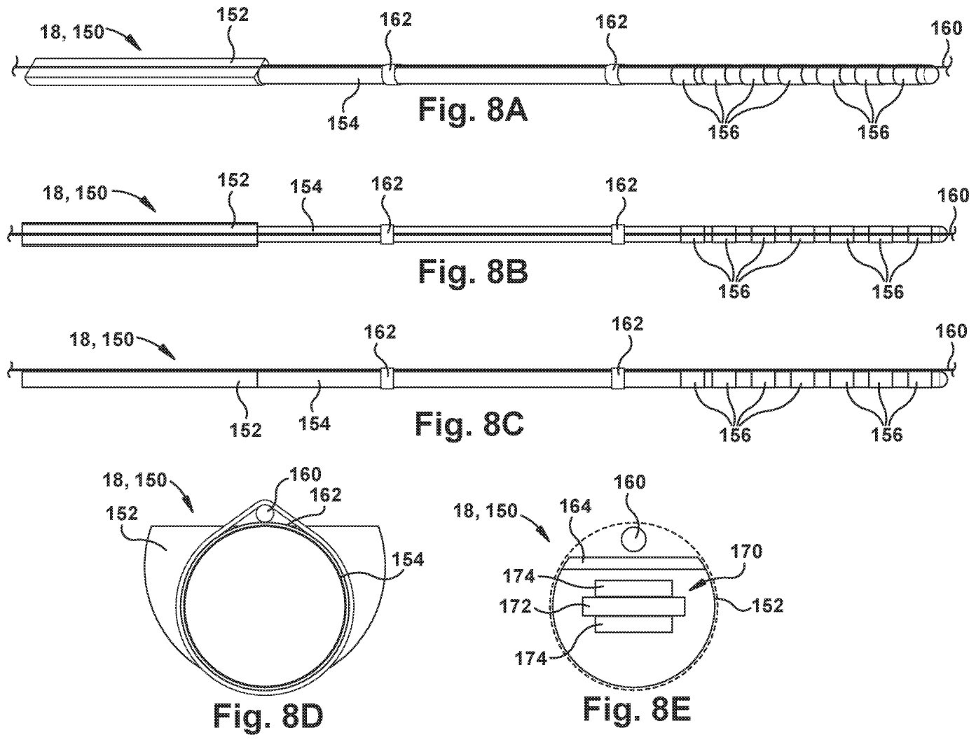

According to another aspect, the neurostimulator 18 can be configured to be used in conjunction with a guidewire for delivering the neurostimulator during implantation without requiring a separate implantation device, such as a needle or tube. An example of this is shown in FIGS. 8A-8E. In these figures, a neurostimulator 150, including an implant body 152 and a lead 154, is outfitted with a guidewire 160. The stimulator 150 can be a one-piece stimulator, such as the stimulator 50 of FIGS. 6A-6D, or a two-piece neurostimulator, such as the stimulator 100 of FIGS. 7A-7E. In the case of the one-piece neurostimulator 50, the guidewire 160 can be connected to the implant body 52 and/or the lead 54. In the case of the two-piece neurostimulator 50, the guidewire 160 can be connected to only the lead 54 and used to deliver only the lead.

The implant body 152 of the neurostimulator 150 can have a flattened configuration so that the guidewire 160 can pass over and extend along the lead 154 with minimal bending. Because of this, the guidewire 160 can be used to deliver the lead 154 alone (two-piece neurostimulator configuration) or along with the implant body 152 (one or two-piece neruostimulator configuration). Holders 162 in the form of loops or straps can be used to help secure the guidewire 160 to the lead 154.

As shown in FIG. 8E, the implant body 152 can include a cover or lid 164 that forms the flattened portion along which the guidewire 160 extends. The cover 164 can be removable to provide access to an interior 170 of the implant body 152, which houses the various components described above (e.g., power portion 30, telemetry portion 32, sensor portion 34, electronics portion 36, etc. See FIG. 5A). These components can include, for example, a printed circuit board (PCB) 172 dedicated to the antenna, power, telemetry, sensor, and electronic components, with ceramic IC packages 174 mounted on one or both sides of the PCB. This structure and these components can be included in any of the sensor configurations of FIGS. 6A-8E.

### Wireless Power Transfer Configuration

Regardless of the particular configuration of the neurostimulator 18 and the methods used to implant the device, the wireless power transfer from the remote transducer 20 to the neurostimulator has several characteristics that can be the same or similar across the platform. For example, antenna excitation power can be ≤1 watt, and the Equivalent Isotropically Radiated Power (EIRP) can be ≤4 watts (W), per FCC guidelines. The remote transducer 20 can generate a 3D electric field for a power transfer of 25-35 milliwatts (mW) between the transducer and stimulator coils/antennas. The frequency of the wireless power transfer can be selected from the frequencies set forth in the following table:

| Frequency | | Conventional Use | Standards |
|---|---|---|---|
| 2.400-2.4835 | GHz | ISM Band (max 4W EIRP) | 802.11/11b |
| 902-928 | MHz | ISM Band (Used by GSM in most | |
| | | countries) | |
| 5.800-5.925 | GHz | ISM Band | |
| 5.15-5.25 | GHz | UNII (Unlicensed - National | 802.11a |
| | | Information Infrastructure) max. | |
| | | 200 mW EIRP | |
| 1-20 | MHz | Wireless charging applications | Qi |
| 126 | kHz | Inductive Resonant Magnetic | |
| | | Coupling | |
| 2-10 | GHZ | Microwave RF Power Transfer | |

Current magnetic wireless power transfer technologies utilize inductive resonant magnetic coupling (126 kHz), which is effective, but is less efficient, can make coil form factors difficult to optimize, and can produce issues with coil directivity, alignment, and orientation, which can make coupling difficult. Microwave RF power transfer technology, which is highly efficient, has a small form factor antenna, and does not exhibit directivity issues, can also be implemented.

For the neurostimulator 18, in order to maintain the small diameter, needle-based implantation capability, the antenna can have a printed circuit board (PCB) configuration, a ferrite rod configuration, a helical coil configuration, or a circular loop configuration.

### Remote Transducer Form Factor

The remote transducer 20 is not limited to a hand-held form factor. The implant body/lead configuration of the neurostimulator allows the electrodes 38 to be positioned at a desired location in the patient and the implant body can be positioned and oriented with the antenna close to the skin surface in a position selected to optimize alignment with, and signal reception from, the remote transducer 20. Because of this, the remote transducer 20 can have one of a variety of form factors, and the neurostimulator 18 can be configured to position the antenna for receiving a stimulation control signal from the particular form factor that is chosen. Examples of some of these form factors are illustrated in FIGS. 9A-9L.

Referring to FIG. 9A, one form factor of the remote transducer 20 can comprise a patch 200 that is adhered to the patient's skin at the location of the implant body of the neurostimulator 18, adjacent the stimulator antenna. This is also described above with reference to FIGS. 4A and 4B. The patch 200 can be battery powered (rechargeable or disposable) and can communicate with a smart device, such as a smartphone 202, via a short range radio communication protocol, such as Bluetooth. Through this communication, the patch 200 can be programmed with settings, patient information, operating parameters, therapy regimens, etc. Operation of the patch 200 can be controlled via an application installed on the smartphone.

Another form factor of the remote transducer 20 can comprise a headset 210, two of which are illustrated in FIGS. 9B and 9C. The headset 210 includes a band 212 for extending over the top of the patient's head, and an arm 214 that extends forward from the area of the patient's ear toward the nose. The arm 214 can be configured to extend around the left or right side of the patient's head. The arm 214 can contain the antenna for powering the neurostimulator 18 and can be configured to position the antenna in a desired position relative to the stimulator antenna. The headset 210 can be battery powered (rechargeable or disposable) and can communicate with a smart device, such as a smartphone, via a short range radio communication protocol, such as Bluetooth. Through this communication, the headset 210 can be programmed with settings, patient information, operating parameters, therapy regimens, etc. Operation of the headset 210 can be controlled via an application installed on the smartphone. Alternatively, the headset 210 can be self-contained, including all of the hardware and software necessary to control operation of the stimulator. In this instance, programming the headset 210 can be performed via a computer, such as a PC, via a wired or wireless connection.

Referring to FIG. 9D, another form factor of the remote transducer 20 can comprise a compact earpiece 220 that includes a band 222 for extending around the patient's ear and a body 224 that extends forward from the patient's ear. The earpiece 220 can be configured to be connected to the patient's left or right ear. The body 224 can contain the antenna for powering the neurostimulator 18 and can be configured to position the antenna in a desired position relative to the stimulator antenna. The earpiece 220 can be battery powered (rechargeable or disposable) and can communicate with a smart device, such as a smartphone, via a short range radio communication protocol, such as Bluetooth. Through this communication, the earpiece 220 can be programmed with settings, patient information, operating parameters, therapy regimens, etc. Operation of the earpiece 220 can be controlled via an application installed on the smartphone. Alternatively, the earpiece 220 can be self-contained, including all of the hardware and software necessary to control operation of the stimulator. In this instance, programming the earpiece 220 can be performed via a computer, such as a PC, via a wired or wireless connection.

Referring to FIGS. 9E and 9F, another form factor of the remote transducer 20 can comprise an extended earpiece 230 that includes a band 232 for extending around the patient's ear, a body 234 that extends from the patient's ear, and an antenna arm 236 that extends forward from the body. The earpiece 230 can be configured to be connected to the patient's left or right ear. The antenna arm 236 contains the antenna for powering the neurostimulator 18 and can be configured to position the antenna in a desired position relative to the stimulator antenna. The earpiece 230 can be battery powered (rechargeable or disposable) and can communicate with a smart device, such as a smartphone, via a short range radio communication protocol, such as Bluetooth. Through this communication, the earpiece 230 can be programmed with settings, patient information, operating parameters, therapy regimens, etc. Operation of the earpiece 230 can be controlled via an application installed on the smartphone. Alternatively, the earpiece 230 can be self-contained, including all of the hardware and software necessary to control operation of the stimulator. In this instance, programming the earpiece 230 can be performed via a computer, such as a PC, via a wired or wireless connection.

Referring to FIGS. 9G and 9H, another form factor of the remote transducer 20 can comprise a handheld remote controller 240 that is simplified to include a single button 242 for initiating stimulation. This single button configuration allows the patient to self-apply stimulation therapy quickly and easily. The remote controller 240 includes a body 244 that includes indicia 246 on the side opposite the pushbutton 242 that coincides with the antenna and guide the patient-user on proper positioning. The remote controller 240 can be battery powered (rechargeable or disposable) and can communicate with a smart device, such as a smartphone, via a short range radio communication protocol, such as Bluetooth. Through this communication, the remote controller 240 can be programmed with settings, patient information, operating parameters, therapy regimens, etc. Alternatively, the remote controller 240 can be self-contained, including all of the hardware and software necessary to control operation of the stimulator. In this instance, programming the remote controller 240 can be performed via a computer, such as a PC, via a wired or wireless connection.

Referring to FIG. 9J, another form factor of the remote transducer 20 can comprise a headband 250. The headband 250 is configured to extend around the patient's head in various locations—over the top, around the back, and even around the lower part of the head and neck. One leg of the headband 250 can contain the antenna for powering the neurostimulator 18. The antenna can be positioned in a desired position relative to the stimulator antenna by adjusting how the headband 250 extends around the patient's head. Additionally, since the headband 250 has a single band configuration, it is ambidextrous in that the antenna can be positioned on either side of the patient's head. The headband 250 can be battery powered (rechargeable or disposable) and can communicate with a smart device, such as a smartphone, via a short range radio communication protocol, such as Bluetooth. Through this communication, the headband 250 can be programmed with settings, patient information, operating parameters, therapy regimens, etc. Operation of the headband 250 can be controlled via an application installed on the smartphone. Alternatively, the headband 250 can be self-contained, including all of the hardware and software necessary to control operation of the stimulator. In this instance, programming the headband 250 can be performed via a computer, such as a PC, via a wired or wireless connection.

Referring to FIG. 9L, another form factor of the remote transducer 20 can comprise eyeglasses 260. In this form factor, the antenna for powering the neurostimulator 18 can be positioned in/on the eyeglasses frame—the temples, the earpiece, the bridge, the nosepads, the rims, or a combination thereof. The antenna can thus be positioned in a desired position relative to the stimulator antenna by adjusting where on the eyeglasses 260 the antenna is positioned and also where in the patient the implant body is located. The eyeglasses 260 can be battery powered (rechargeable or disposable) and can communicate with a smart device, such as a smartphone, via a short range radio communication protocol, such as Bluetooth. Through this communication, the eyeglasses 260 can be programmed with settings, patient information, operating parameters, therapy regimens, etc. Operation of the eyeglasses 260 can be controlled via an application installed on the smartphone. Alternatively, the eyeglasses 260 can be self-contained, including all of the hardware and software necessary to control operation of the stimulator. In this instance, programming the eyeglasses 260 can be performed via a computer, such as a PC, via a wired or wireless connection.

Operation of the remote transducer 20 can be initiated via the transducer itself, e.g., via buttons or switches on the transducer, via a smart device, such as a smartphone, or via a remote control device. For example, operation of the remote transducer 20 can be initiated by remote control devices, such as a foot pedal 270 (FIG. 9I) or a key fob remote 280 (FIG. 9K), each of which can be configured to communicate with the remote transducer 20 via a short range radio communication protocol, such as Bluetooth.

### Implantation Methods

There are several surgical approaches that may be used to deliver a neurostimulator 18 into the PPF 14 via the delivery device 26 (see FIGS. 3A-3B). One approach is a gingival-buccal approach, which is described in U.S. Pat. No. 9,211,133 to Papay and describes a therapy delivery device that has a curvilinear shape. Another approach is a trans-oral approach, with a dental needle up to the sphenopalatine foramen through the posterior palatine canal (see U.S. Pat. No. 8,229,571 to Benary et al.). Another approach is a trans-nasal approach. Yet another approach is a lateral approach with a straight needle to the PPF 14 through the infratemporal fossa (see U.S. Pat. No. 6,526,318 to Ansarinia). A further approach includes an infrazygomatic approach, in which the skin entry is at a site overlying the PPF 14, just inferior to the zygoma and anterior to the mandible. Other routes through the mouth and outer skin of the face are described in M. duPlessis et al., Clinical Anatomy 23 (8, 2010):931-935, Micah Hill et al., Operative Techniques in Otolaryngology 21 (2010):117-121, and M I Syed et al., Radiology of Non-Spinal Pain Procedures. A Guide for the Interventionist. Chapter 2. Head and Neck. pp. 5-42 (Heidelberg: Springer, 2011).

The SPG 10 can be localized using at least one scanning apparatus, such as a CT scan or fluoroscope. Further details of the localization procedure are disclosed in U.S. Pat. No. 6,526,318 to Ansarinia.

The entry point for the insertion of the delivery device 26 can be located in the coronoid notch between the condylar and coronoid processes of the ramus of the mandible. Once the entry point is localized, the skin overlying the entry point can be shaved and prepared with antiseptic solution. A 25-gauge needle can be used to inject a subcutaneous local anesthetic (e.g., 2 cc of 2% lidocaine) into the skin and subcutaneous tissues overlying the entry point. In addition to the local anesthetic, the patient may be given intravenous sedation and prophylactic antibiotics prior to commencement of the implantation procedure, if desired. In this manner, the patient can receive the local anesthetic for pain and comfort while still being to detect paresthesia so that they can assist by giving feedback during lead delivery and placement.

The delivery device 26 can be inserted at the entry point and advanced between the coronoid process and the condylar process of the ramus of the mandible towards the PPF 14 (FIGS. 3A-3B). The delivery device 26 can be slowly advanced in the medial fashion perpendicular to the skin in the anterior-posterior (transverse) plane along the direction of the x-ray beam of the fluoroscope until it enters the PPF 14. Once the delivery device 26 is positioned according to whether implantation is desired on or adjacent the SPG 10, a stylet (not shown) is withdrawn from the delivery device 26. An electrode (not shown) can then be placed within the central channel of the delivery device 26 and used to test the placement of the delivery device. Next, the neurostimulator 18 can be advanced to the distal tip of the delivery device 26 to place the neurostimulator on or proximate to the target neural structure (e.g., the SPG 10).

In one example, the neurostimulator 18 can be implanted in the patient without penetrating the cranium of the patient. In another example, the neurostimulator 18 can be implanted in the patient without penetrating the palate and/or without entering the nasal cavity of the patient.

The neurostimulator 18 is configured so that it can be implanted using a standard surgical needle. For the one-piece stimulator 50 of FIGS. 6A-6D, the implant body 52, having the largest diameter, dictates the size of the needle that can be used for implantation. For example, the stimulator 50 can be implanted using a 14 gauge surgical needle.

For the two-piece stimulator 100 of FIGS. 7A-7E, the implant body 102 and lead 104 can be delivered separately, so their individual sizes dictate the size of the needle, tube, or other delivery device used for implantation. The lead 104, having a small diameter compared to the lead body 102 can be implanted with a smaller sized delivery device. For example, the lead 104 can be implanted using an 18 gauge surgical needle, whereas the implant body can be implanted using a 14 gauge surgical needle. The two-piece stimulator configuration offers some advantages in this regard. The lead 104 is implanted first, deeper into the patient's anatomy, with a high degree of precision. The smaller sized needle is more dexterous and less invasive, so it is better-suited for delivering the lead 104.

Additionally, since the implant body 102 can be implanted with less precision and closer to the skin surface, its delivery is better suited for the larger 18 gauge needle. Since the implant body 102 can be implanted closer to the surface, and since the lead 104 is flexible/bendable, the implant body can be positioned, oriented, and aligned in an ideal manner for communicating with the remote transducer 20. This position and orientation can be tailored to complement the chosen form factor of the remote transducer 20 (see FIG. 9).

The neurostimulator 18, or portions thereof, can also be implanted using a technique known as a Seldinger technique. According to this technique, the desired tissue is punctured with a sharp hollow needle called a trocar, with ultrasound/image guidance if necessary. A round-tipped guidewire is then advanced through the lumen of the trocar and through the tissue to a desired location relative to the SPG, and the trocar is withdrawn. A tube, such as a sheath, cannula, etc., is then passed over the guidewire into the patient to the desired location. Once the tube is positioned, the guidewire is withdrawn. The neurostimulator 18 can then be delivered to the SPG through the tube, and the electrodes 38 can be positioned at the desired location. Once the neurostimulator 18 is secured at the desired position and location, the tube can be removed, leaving the neurostimulator in place.

The Seldinger technique can be advantageous for implantation of the two-piece stimulator 100 of FIGS. 7A-7E. Using this approach, the lead 104 can be implanted first using the Seldinger technique. Once the lead 104 is delivered and secured at the desired position and location, and the delivery tube is removed, the implant body 102 can be implanted using another delivery technique, such as via surgical needle, tube, cannula, guidewire, etc., and connected to the lead 104 via the connector 116 to complete the stimulator 100.

With regard to the stimulator 150 of FIGS. 8A-8E, the stimulator can be implanted with the assistance of the guidewire 160, which is attached to the implant body 152 and/or the lead 154. Using this approach, the stimulator 150 can be delivered via the guidewire alone, thus eliminating the need for a delivery needle or tube. The guidewire 160, with the stimulator 150 attached, can be introduced into the patient's anatomy, e.g., through an incision or using a trocar, and guided (with ultrasound/image guidance, if desired) to the implant site. Once the stimulator 150 is positioned and secured at the implant site, the guidewire can be removed, leaving the stimulator in place.

The delivery and implantation methods described herein can also be combined. For example, for a two-piece configuration of the stimulator 150, the lead 154 can be delivered via the guidewire 160, and the implant body 152 can be delivered using a needle. As another example, for a two-piece configuration of the stimulator 150, the lead 154 can be delivered using the Seldinger technique, and the implant body 152 can be delivered using a needle.

### Neurostimulation Methods

Once implanted, the remote transducer 20 can be brought into contact (or close contact) with the head of the patient so that the remote transducer is within close proximity (which can range from approximately 2 centimeters to approximately 10 meters) to the implanted neurostimulator 18. Where the remote transducer 20 comprises a patch, for example, a skin-contacting surface of the patch can be brought into direct contact with the cheek of the patient, immediately adjacent the location of the implanted neurostimulator 18.

The remote transducer 20 can be activated (FIGS. 4A-4B). Activation of the remote transducer 20 causes the neurostimulator 18 to deliver an electrical signal to the target neural structure (e.g., the SPG 10). In some instances, electrical energy can be applied to the target neural structure (e.g., the SPG 10) for a time and in an amount insufficient to cause a lesion on the target neural structure. In other instances, electrical energy can be delivered to the target neural structure in any of several forms, such as biphasic charge-balanced pulses having a frequency of about 1-1000 Hz (e.g., 5-200 Hz), a pulse-width of about 0.04-2 ms, a current of about 0.05-100 mA (e.g., 0.1-5 mA), and a voltage of about 1-10 V. In addition, electrical modulation can be controllable such that either anodic or cathodic stimulation may be applied. Electrical energy may be delivered continuously, intermittently, as a burst in response to a control signal, or as a burst in response to a sensed parameters, such as increased SPG 10 neural activity. The electrical parameters may also be adjusted automatically based on a control signal, based on sensed parameters, or by selection by the patient (e.g., using the personal electronic device). The electrical energy can be applied to the target neural structure for a time and in an amount sufficient to treat the medical condition.

In one example neurostimulation method, the neurostimulator 18 can be used to treat a migraine headache in a manner such that the therapy is transparent to the patient. To do so, stimulation parameters can be programmed into the neurostimulator 18 so that patient does not feel the paresthesia that can accompany neurostimulation. According to this method, a patient can be treated for a headache by controlling non-paresthesia stimulation of autonomic system, such as the SPG by implanting lead having at least one electrode at a target position proximate to the SPG of the patient. A non-paresthesia stimulation waveform can be delivered to the at least one electrode based on a therapy parameter set (TPS). The stimulation waveform can include a series of pulses configured to excite at least one of A-delta fibers or C-fibers of the SPG of the patient. Sensory action potential (SAP) signals can also be sensed. The method can include iteratively delivering the non-paresthesia waveform and sensing the SAP signals while changing at least one parameter from the TPS. The SAP signals can be analyzed to obtain SAP activity data associated with the TPS for at least one of an SAP C-fiber component or an SAP A-delta fiber component. Through this analysis, a collection of SAP activity data associated with multiple therapy parameter set can be obtained. One or more parameters for the TPS can be selected based on the collection of SAP activity data. The pulse generator of the neurostimulator can be programmed to deliver stimulation to the SPG according to the TPS, and the neurostimulator can be activated so that the pulse generator delivers electrical stimulation to the patient according to the programmed TPS.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

We claim:

1. An implantable neurostimulator comprising:
a lead comprising a plurality of electrodes at a distal end;
an implant body comprising electronics for controlling operation of the electrodes; and
an electrical connector for establishing an electrical connection between the electronics and the electrodes;
wherein the implant body comprises a first portion of the electrical connector and the proximal end of the lead comprises a second portion of the electrical connector, the first and second portions of the electrical connector being connectable to establish the electrical connection between the electronics and the electrodes; and
wherein the lead is configured for initial implantation in the patient and the implant body is configured for subsequent implantation in the patient, and wherein the electrical connector is configured so that the connection of the first and second portions can be performed with the implant body and the lead positioned at a surgical site in the patient.

2. The implantable neurostimulator recited in claim 1, wherein the electrical connector is a plug-in connector configured so that the electrical connection can be made by pressing together the first and second portions.

3. The implantable neurostimulator recited in claim 2, wherein the first portion of the electrical connector has a stepped female configuration and the second portion of the electrical connector has a stepped male configuration.

4. The implantable neurostimulator recited in claim 1, wherein the second portion of the connector is configured for a connection via wires to an external controller, wherein the external controller is configured to energize the electrodes during implantation in order to obtain feedback for use in positioning the electrodes.

5. The implantable neurostimulator recited in claim 4, wherein the feedback is feedback indicative of sensing paresthesia induced by the electrodes.

6. The implantable neurostimulator recited in claim 1, wherein the lead is configured to pass through an 18 gauge surgical needle.

7. The implantable neurostimulator recited in claim 1, wherein the implant body is configured to pass through a 14 gauge surgical needle.

8. The implantable neurostimulator recited in claim 1, further comprising a remote transducer for providing a wireless signal for powering the stimulator, the remote transducer comprising at least one of a patch, headset, earpiece, extended earpiece, handheld remote controller, headband, and eyeglasses.

9. The implantable neurostimulator recited in claim 8, further comprising a remote controller for controlling operation of the remote transducer, the remote controller comprising a foot pedal or a key fob that communicates wirelessly with the remote transducer.

10. A method for implanting a two-piece neurostimulator comprising an electrode lead and an implant body connectable with the lead to supply power for energizing the electrodes to apply stimulation therapy, the method comprising:
implanting the lead using a Seldinger technique; and
implanting the implant body; and
connecting the implant body to the lead.

11. The method of claim 10, wherein the neurostimulator is implanted via a gingival-buccal approach, a transoral approach, a trans-nasal a lateral approach through an infratemporal fossa of the patient, or an infrazygomatic approach in which the entry site of the neurostimulator is inferior to the zygoma and anterior to the mandible.

12. A method for implanting a two-piece neurostimulator comprising an electrode lead and an implant body connectable with the lead to supply power for energizing the electrodes to apply stimulation therapy to a patient, the method comprising:
attaching a guidewire to the lead;
implanting the lead using the guidewire to navigate through the patient's anatomy and position the electrodes at a desired site in the patient;
removing the guidewire, leaving the lead implanted;
implanting the implant body; and
connecting the implant body to the lead.

13. The method of claim 12, wherein the neurostimulator is implanted via a gingival-buccal approach, a transoral approach, a trans-nasal a lateral approach through an infratemporal fossa of the patient, or an infrazygomatic approach in which the entry site of the neurostimulator is inferior to the zygoma and anterior to the mandible.

14. A method for implanting a neurostimulator comprising an electrode lead and an implant body for supplying power for energizing the electrodes to apply stimulation therapy to a patient, the method comprising:
attaching a guidewire to the neurostimulator;
implanting the stimulator using the guidewire to navigate through the patient's anatomy and position the electrodes at a desired site in the patient; and
removing the guidewire, leaving the stimulator.

15. The method of claim 14, wherein the neurostimulator is implanted via a gingival-buccal approach, a transoral approach, a trans-nasal a lateral approach through an infratemporal fossa of the patient, or an infrazygomatic approach in which the entry site of the neurostimulator is inferior to the zygoma and anterior to the mandible.

16. A method for implanting a two-piece neurostimulator comprising an electrode lead and an implant body connectable with the lead to supply power for energizing the electrodes to apply stimulation therapy to a patient, the method comprising:
anesthetizing the patient using an anesthesia solution that is sufficient for controlling pain but allows the patient to perceive paresthesia from stimulation;
connecting the lead to an external controller that is operable to energize the electrodes to apply stimulation;
surgically implanting the lead while applying stimulation via the electrodes;
querying the patient for feedback regarding perceived paresthesia during while implanting the lead;
using the feedback from the patient to assist in determining a proper position for the lead;
securing the lead in the proper position;
disconnecting the lead from the external controller; and
surgically implanting the implant body and connecting the implant body to the lead.

17. The method of claim 16, wherein the neurostimulator is implanted via a gingival-buccal approach, a transoral approach, a trans-nasal a lateral approach through an infratemporal fossa of the patient, or an infrazygomatic approach in which the entry site of the neurostimulator is inferior to the zygoma and anterior to the mandible.

18. A method for treating a migraine headache in a patient using an implantable neurostimulator, comprising:
programming stimulation parameters into the neurostimulator so that patient does not perceive paresthesia from electrical stimulation of the sphenopalatine ganglion (SPG);
implanting the neurostimulator so that a lead of the stimulator having at least one electrode is at a target position proximate to the SPG of the patient;
delivering a non-paresthesia stimulation waveform to the at least one electrode based on a therapy parameter set (TPS), the stimulation waveform including a series of pulses configured to excite at least one of A-delta fibers or C-fibers of the SPG of the patient;
sensing sensory action potential (SAP) signals of the patient;
iterating the steps of delivering the non-paresthesia stimulation waveform and sensing the SAP signals while changing at least one parameter from the TPS;
analyzing the SAP signals to obtain SAP activity data associated with the TPS for at least one of an SAP C-fiber component or an SAP A-delta fiber component to obtain a collection of SAP activity data associated with multiple therapy parameter set;
selecting one or more parameters for the TPS based on the collection of SAP activity data;
programming a pulse generator of the neurostimulator to deliver electrical stimulation to the SPG according to the TPS; and
activating the neurostimulator so that the pulse generator delivers electrical stimulation to the patient according to the programmed TPS.
